(19) United States
(12) Patent Application Publication
(10) Pub. No.: US 2008/0056439 A1
(43) Pub. Date: Mar. 6, 2008

(54) Multi-use CT scanning apparatus

(75) Inventors: Tim Thompson, Santa Fe, NM (US); David Phillips, Los Alamos, NM (US)

Correspondence Address: HARVEY CONSULTING PLLC, PO BOX 6568, NIRMAN, OK, 73072, US

(73) Assignee: IMTEC Imaging, LLC

(21) Appl. No.: 11/443,975

(22) Filed: May 31, 2006

Related U.S. Application Data
(60) Provisional application No. 60/727,314, filed on Oct. 17, 2005.

(51) Int. Cl. A61B 6/03

(52) U.S. Cl. 378/15

(57) ABSTRACT

The present invention relates generally to the field of CT and X-ray scanning units. More specifically, the invention relates to scanning unit with the capability to scan impressions and patients at the same location. Even more specifically, the invention involves a method of using data and images received from the same scanning unit to fabricate a surgical stint or guide device that snap fits onto a preparation site, and further allows a dental practitioner or a lab to prepare permanent prosthetics using the data and images from the same scanning unit. Even more specifically, the invention relates to a method of allowing the clinician to place high-fidelity teeth back into the correct location within the jaw taus and create models and treatment plans using hi-fidelity 3D images. Additionally, the invention relates to attaching an imaging capturing device (such as a digital camera) that allows the transmission of digital images or data from the capturing device to reconstructing software that reconstructs the 3D volume of the head topography to superimpose over the CT scan of head. The reconstructing software may reconstruct data from the captured digital images or data, from the data received from scanning patients and from scanning impressions. Thus, in an industry that previously required three different machines to scan impressions, the interior of the head and the topography of the head. SLD printers can print out a wax model utilizing crowns and bridges that is accurate and ultimately replaces lab technicians.

This application claims the priority of the following U.S. provisional patents: 1) U.S. Pat. Appl. Ser. No. 60/727,314,2) U.S. patent application Ser. No. 10/686,520.

### TECHNICAL FIELD OF THE INVENTION

The present invention relates generally to the field of CT and X-ray scanning units. More specifically, the invention relates to scanning unit with the capability to scan impressions and patients using the same machine. Even more specifically, the invention involves a method of using data and images received from the same scanning unit to fabricate a surgical stint or guide device that snap fits onto a preparation site, and further allows a dental practitioner or a lab to prepare permanent prosthetics using the data and images from the same scanning unit. Even more specifically, the invention relates to a method of allowing the clinician to place high-fidelity teeth back into the correct location within the jaw taus and create models and treatment plans using hi-fidelity 3D images. Additionally, the invention relates to attaching an imaging capturing device (such as a digital camera) that allows the transmission of digital images or data from the capturing device to reconstructing software that reconstructs the 3D volume of the head topography to superimpose over the CT scan of head. The reconstructing software may reconstruct data from the captured digital images or data, from the data received from scanning patients and from scanning impressions. Thus, to accomplish what previously required three different machines—to scan impressions, the interior of the head and the topography of the head—the present invention capably addresses all three tasks in one machine.

### BACKGROUND

It is well known in the dental field that computer tomography (CT) scanning units offer advantages for many different types of dental applications. Scanning units addressing the applications of taking dental impression scans and of mandible/maxilla scans have been disclosed only in separate scanning units. Therefore in the dental field, CT scanning machines exist for specialized and somewhat limited purposes.

The images acquired from a CT scan of the mandible/maxilla region are known to aid dental practitioners in developing a treatment plan involving the placement of dental implants. A CT scan of the mandible/maxilla region may reveal bone ridge location and dimensions. A CT scan will also reveal the location of mental foramen, the nerve that practitioners must avoid during the placement of dental implants. Equipped with images produced from a CT scan (filtered through appropriate reconstructive software), a dental practitioner may map out the exact locations and angles for inserted dental implants to engage as much bone as necessary while avoiding the mental foramen.

The practice of taking impressions for capturing a dental patient's occlusion and dentition is also well known in the dental arts. For the purposes of dental implantation, an impression enables a practitioner or a lab to create a model for implant placement and eventually prosthetic device or devices tailored to dental implants that are placed within the dental patient's jawbone.

Initially, impressions may be utilized in a practitioner's treatment plan to develop surgical stints for guidance in placing implants, especially for dental patients who are only partially edentulous. A CT scan of the patient's mandible/maxilla region is helpful to determine the bony ridge dimensions and location of the mental foramen so that implants may be placed effectively and safely. Indeed, such a treatment plan for placing implants is becoming the accepted standard of care in dental implantation.

After the implant placement, the practitioner or lab may also use the patient's impression to create mandible/maxilla models, the models being primarily valuable in making crowns and bridges for prepped natural teeth to be cemented upon prepped abutments. The practitioner or lab may further use the model created the model make custom abutments or removable prosthetics for existing appliances, such as a bar.

Many dental practitioners may also wish to utilize CT scans for the purpose of imaging an impression. As seen in U.S. Pat. No. 6,767,208 ('208), a process is disclosed comprising taking impressions of the teeth, capturing the bite, scanning the impressions using an x-ray source, making a positive of the scans, aligning the scan date, detailing scanned models, setting the final bite, and generating treatment using digital data. '208 is effective for the limited purposed of imaging an impression. If the practitioner's treatment plan included placing dental implants, then a separate scanning machine for conducting a mandible/maxilla scan of the dental patient would be necessary in the current art. Some dental practitioners also wish to merge data sets from a CT scan of their patients with data from those patients' bite plates.

In certain specialized fields of dental and prosthetic practice, it is also helpful for the practitioner to capture the surface of a patient's face. Currently and in the very least, the fields of cranial facial and oral maxillofacial surgery have applications for surface renderings of the face and head to develop treatment and/or surgical plans.

For a practitioner in a dental practice that has limited office and lab space, it is impractical to attempt to operate two or more x-ray scanning units for different applications. Furthermore, dental practitioner in many specialties have a great need for accurately merging data from a CT Scan with that of a facial topographical scan. Therefore, a need exists for dental implantation practitioners for an imaging unit that can independently scan a patient's maxilla/mandible region and an impression, because an impression scan is not desirable. A need further exists for a scanning system that can integrate the data from independent CT scans and impression scans to create a model and a secure surgical stint for exact implant placement. Finally, a need exists for a dental implantation practitioner to reduce the time and patient visits by integrating steps of a treatment plan.

### SUMMARY OF THE INVENTION

In one embodiment of the invention, a scanning apparatus is disclosed capable of scanning impressions, a 3D surface rendering of a dental patient, and a CT scan of the dental patient at the same location, the unit comprising:

a chair for holding and positioning an x-ray target, the x-ray target optionally a dental patient or a dental impression;

an arm that revolves around the x-ray target,

an x-ray source;

a plate for receiving x-rays from x-ray source that travel through the x-ray target,

a digital capturing device to capture a surface rendering of the x-ray target, extending from the arm, and

a computer for reconstructing and integrating data and images resulting from scanning impressions, the surface renderings from the digital capturing device and the x-ray of dental patients; and optionally further comprising a 3D printer for printing out 3D models from the data and images transmitted by the computer.

These and other features, aspects and advantages of the present invention will become better understood with reference to the following drawings, description, and claims. For a better understanding of the invention, its operating advantages and the specific aspects of its uses, reference should be made to the accompanying drawings and descriptive matter in which there are illustrated preferred embodiments of the invention. The foregoing has outlined some of the more pertinent aspects of the invention. These aspects should be construed to be merely illustrative of some of the more prominent feature and applications of the present invention. Many other beneficial results can be attained by applying the disclosed invention in a different manner or by modifying the invention within the scope of the disclosure. Accordingly, a fuller understanding of the invention and the detailed description of the preferred embodiments in addition to the scope of the invention are illustrated by the accompanying drawings.

### BRIEF DESCRIPTION OF THE DRAWINGS

FIG. 1 is a side perspective view of the imaging device during a CT scan.

FIG. 2 is a side perspective view of the imaging device during an impression scan.

FIG. 3 is a top perspective view of the impression holder.

FIG. 4 is a top plan view of the x-ray source, the x-ray transmission, and the flat panel sensor.

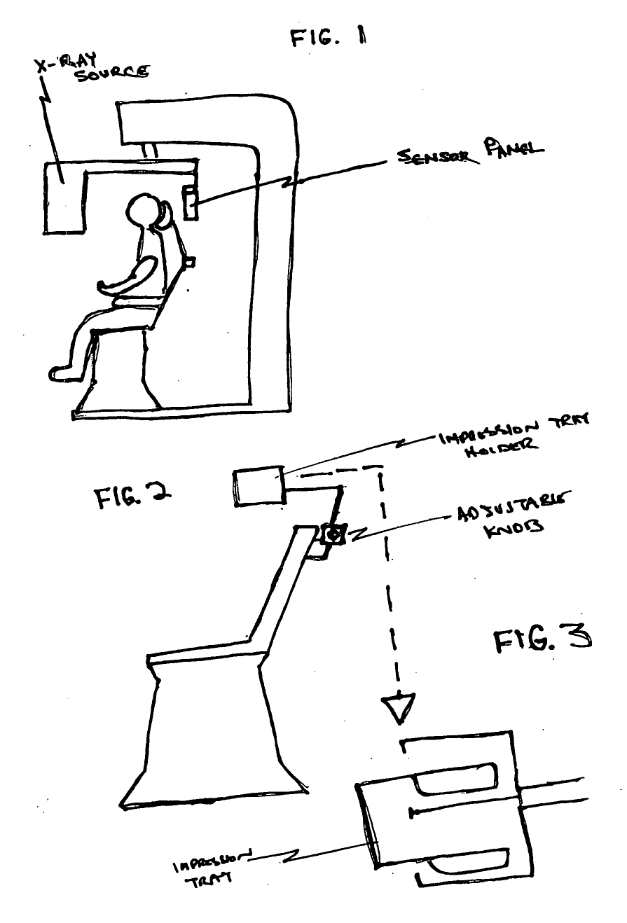

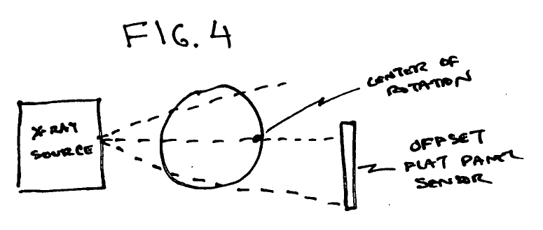

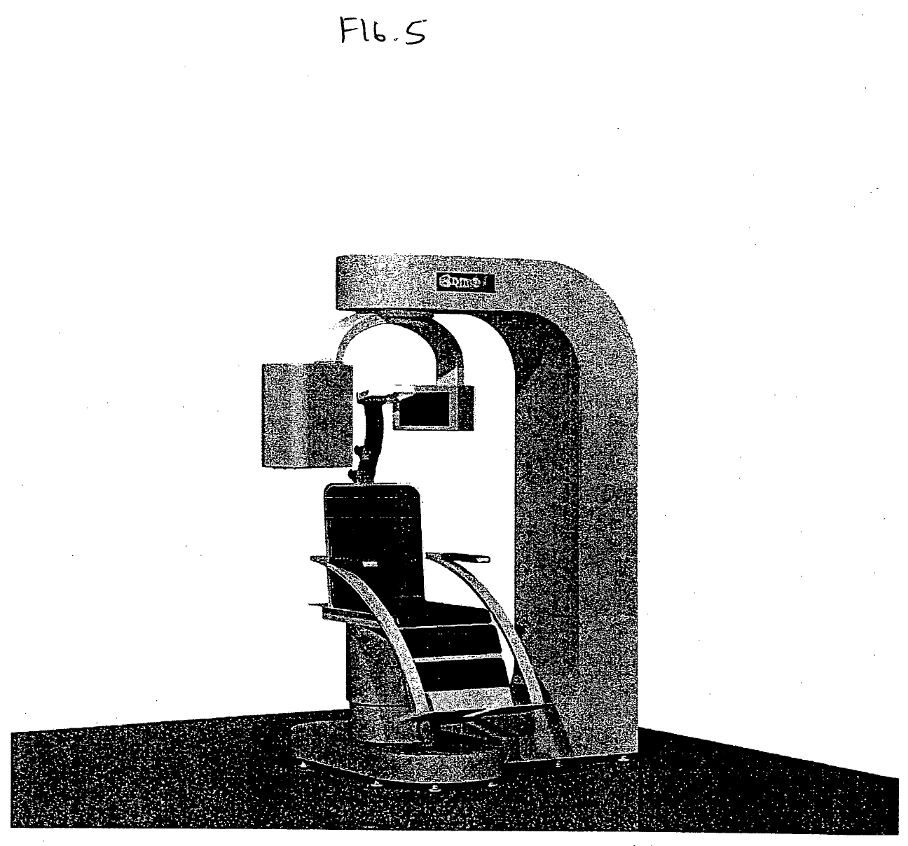

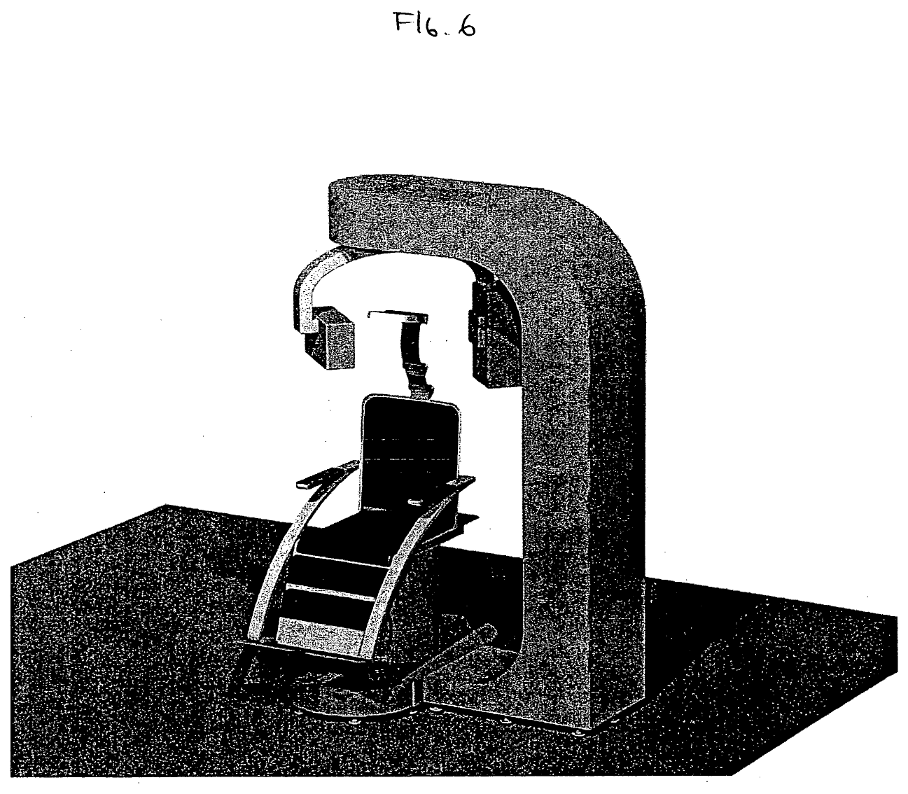

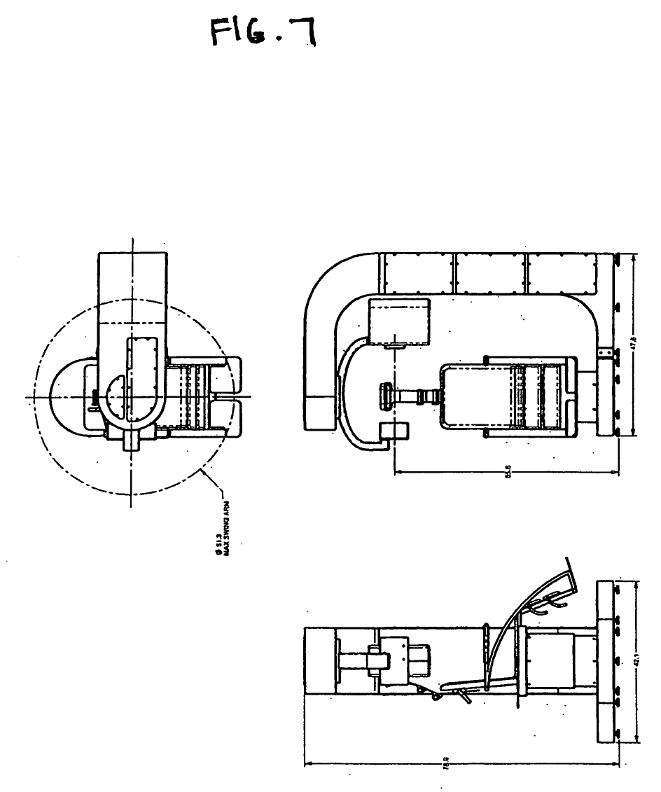

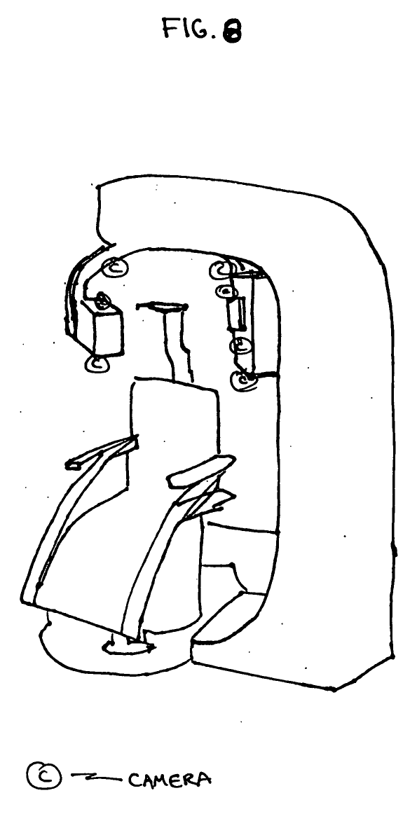

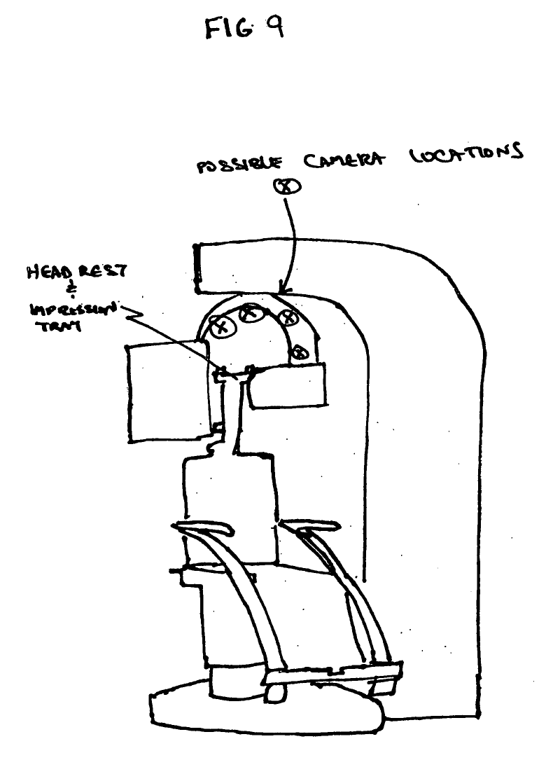

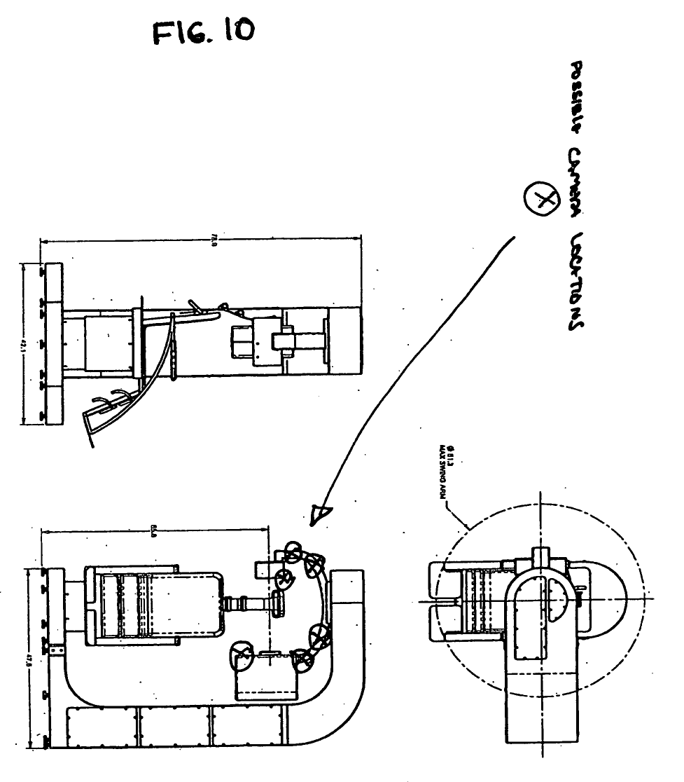

### DETAILED DESCRIPTION OF THE INVENTION

The following detailed description shows the best currently contemplated modes of carrying out the invention. The description is not to be taken in a limiting sense, but is made for the purpose of illustrating the general principles of the invention and the best mode for practicing the invention, since the scope of the invention is best defined by the appended claims.

The method and apparatus of imaging for the purpose of dental implantation treatment plans envisioned by the present invention differs greatly both in function and in structure from past attempts to create imaging systems to address the problems in the industry. The present invention provides the novel advantage of enabling dental practitioners the ability to use the same imaging machine to take imaging scans of dental impressions, of the mandible/maxilla regions of dental patients and of the topographical features in dental patient's faces.

### CT Scanning Protocol for Treatment Plan

In one approach, the inventive CT scanning system may scan the mandible/maxilla of the a dental patient, much like other scanners in the art of CT scanning, and particularly as such in the dental implant industry. The patient may be seated in a chair or stand with a vertical adjustment means to align the patient to a height that is appropriate and enabling of a full scan of the targeted mandible/maxilla region. The CT scan itself operates in a normal fashion. An x-ray source orbits the patient or other target, focusing x-rays through the targeted region. A detector plate positioned opposite the x-ray source receives or detects the x-rays that pass through the patient's targeted head and neck region, collecting and transmitting the resulting data from the x-ray passing through the targeted region. An inventive aspect to the CT scanning system is that the detector plate may be a flat panel sensor, using extended field of view with the flat panel. Software actualizes the data into 3D images. A practitioner may manipulate the images to view different slices of patient's mandible/maxilla region, using the data to develop a treatment plan. For example, the practitioner may use the manipulated images and slices thereof to simulate the placement of dental implants into the patient's jawbone, locating and avoiding the placement of dental implants troublesome areas such as the mental foramen nerve.

Continuing the scanning protocol, an additional scan may be conducted of a dental impression. First, the practitioner should take an impression of the patient's occlusion and dentition, using any materials well known in the industry to capture the patient's bite. Second, the practitioner should mount the impression upon an impression holder on the inventive scanning system and scan the impression without a human in the chair, focusing the x-rays through the impression as the targeted region. The scanning system therefore incorporates an inventive design aspect that allows a practitioner to substitute the scanning target (here, an impression for a patient's mandible/maxilla region). That is, a dental practitioner may effectively and securely place the impression holder upon the chair so that the impression becomes the scanning target. After the impression is scanned, the resulting data is then converted from a negative to a positive image and then integrated into the 3D image generated by the CT scan of the same patient's mandible/maxilla region. The integrated 3D image may be acquired and viewed on a computer and an accompanying display screen.

The resulting integrated data and images from the two scans may be sent from the computer to a fabricating machine or 3D printer for production of products such as 3D models, the 3D models constructed from resilient materials such as plastics or other materials known in the arts that may be printed from a 3D printer. A 3D printer using integrated data and images sent from the computer may produce a 3D model that can be substituted for the conventional stone model currently used in the dental arts. Using the 3D models, surgical splint guides may be forged in the manner generally known in the arts to allow the practitioner to accurately place dental implants in the desired angle and depth according to the treatment plan conceived from the patient's initial mandible/maxilla scan.

The integrated data and images may also be sent to a lab for immediate construction of long-term prosthetics that will interface with the dental implants. Because of the inherent accuracy of using the integrated data and images from the two scans, the long-term prosthetics components may be constructed and delivered back to the practitioner before the dental implants are even placed, enabling the practitioner to immediately load the implants with prosthetic components, if the treatment plan allows.

### Topographical Scan

In another embodiment of the invention, a topographical scanning device may be affixed to the inventive machine. By example and not limitation, the device may utilize laser, visible light patterns or any other technology that will capture topographical features of an object. The topographical device may be a digital camera, film camera, video camera or any other device known in the industry to capture a topographical surface of an object. More specifically, the device may affix to a gantry of the inventive machine to allow the device to capture the topographical features of the object from the same angles or vantage points as the x-ray source of the CT scanning system describe in the above CT SCANNING PROTOCOL FOR TREATMENT PLAN. Therefore, it is foreseen by the present invention that the device may capture as little as a single image from a single angle to a continuous capture of 360 degrees about the object.

As the topographical scanning device captures data sets of a surface rendering of the topographical features of the object, those data sets may be combined with other data sets from the CT Scanning system. Because the data sets from the surface rendering of the topographical features of the object are captured from the same angle, the merging of those two or more data sets will give a profile of the object that is much more accurate than if the data sets were taken from independent sources.

By example and not limitation, applications for the topographical scan may include prosthodontists, oral surgeons and other health care aestheticians who wish to visualize topographical data such as a visible smile line, eyes, and topographical center of a patient's face as opposed to an anatomical center of the face, the difference between which may only be a few all-important millimeters. CT scans do not always reveal such topographical data that ultimately controls the aesthetics of reconstructive and cosmetic surgery. The present inventive machine provides a solution to those professionals needing data sets from CT scans and from topographical scans that merge accurately. The professionals can take topographical scans as often as needed from as many views as needed without exposing the patients to radiation more than necessary, aiding in pre-operation and post-operation case presentations.

In other embodiments of the invention, it is foreseen that this multi-faceted and innovative machine may be used in contexts other than that of strictly dental and cranial-facial applications. With the inventive machine and its accompanying software and computer processing unit, one may scan or capture the exterior and the interior of any object of interest. That the reconstructed image renderings of the object of interest manifest an amount of coloration and layered texture that have not been disclosed in the arts is also considered an inventive aspect of the present application. By adding the additional layer of taking a CT scan of another object than the initial object of interest and integrating it into the reconstructed image rendering, another inventive aspect may be seen. For example, the general ability to scan objects of interest topographically and in vitro in all medical and veterinary arts may be accomplished with the current invention. Applications with the current invention may also made to objects of interest outside of the above arts, such as where objects of interest are scanned to examine its contents for safety concerns (such as explosives or other anti-terrorism concerns).

As has been demonstrated, the present invention provides advantageous techniques for a scanning patients and impressions that provide a single scanning unit with features providing more accurately-fabricated products such as surgical stints and prosthetics components also styled according to the invention. The present invention provides the advantage of acquiring different sets of data from geometrically congruent angles; that is, both the x-ray source and the topographical imager are mounted from the same angle from the gantry or arm. While the preferred embodiments of the present invention have been described, additional variations and modifications in those embodiments may occur to those skilled in the art once they learn of the basic inventive concepts. Therefore, it is intended that the appended claims shall be construed to include both the preferred embodiment and all such variations and modifications as fall within the spirit and scope of the invention.

We claim:

1. A scanning apparatus capable of scanning impressions, a 3D surface rendering of a dental patient, and a CT scan of the dental patient at the same location, the unit comprising:
a scanning apparatus is disclosed capable of scanning impressions, a 3D surface rendering of a dental patient, and a CT scan of the dental patient at the same location, the unit comprising:
a chair for holding and positioning an x-ray target, the x-ray target optionally a dental patient or a dental impression;
an arm that revolves around the x-ray target,
an x-ray source;
a plate for receiving x-rays from x-ray source that travel through the x-ray target,
a topographical imager to capture a surface rendering of the x-ray target, extending from the arm, and
a computer for reconstructing and integrating data and images resulting from scanning impressions, the surface renderings from the digital capturing device and the x-ray of dental patients; and optionally further comprising a 3D printer for printing out 3D models from the data and images transmitted by the computer.

2. The scanning apparatus from claim 1, the apparatus further comprising:
a 3D printer for printing out 3D models from the data and images acquired by the computer.

3. The scanning apparatus in claim 1, the plate further comprising:
a flat panel sensor, the sensor being off-set and using an extended field of view from transmissions sent by the x-ray source.
